(19) United States
(12) Patent Application Publication
(10) Pub. No.: US 2025/0082330 A1
(43) Pub. Date: Mar. 13, 2025

(54) NON-WOVEN GRAFT MATERIALS FOR NERVE REPAIR AND REGENERATION

(71) Applicant: Acera Surgical, Inc., St. Louis, MO (US)

(72) Inventors: Matthew R. MacEwan, St. Louis, MO (US); Lily Jeng, St. Louis, MO (US); Abdolrasol Rahimi, St. Louis, MO (US); Manisha Jassal, West Warwick, RI (US); Tamas Kovacs, St. Louis, MO (US)

(21) Appl. No.: 18/946,153

(22) Filed: Nov. 13, 2024

Related U.S. Application Data
(63) Continuation of application No. 17/903,918, filed on Sep. 6, 2022, now Pat. No. 12,167,853.
(60) Provisional application No. 63/241,366, filed on Sep. 7, 2021.

(51) Int. Cl. A61B 17/11; A61L 27/18; A61L 27/22; A61L 27/24; A61L 27/54; A61L 27/56; A61L 27/58

(52) U.S. Cl. CPC A61B 17/1128; A61L 27/18; A61L 27/225; A61L 27/24; A61L 27/54; A61L 27/56; A61L 27/58; A61L 2300/412; A61L 2300/604; A61L 2430/32

(57) ABSTRACT

Disclosed herein are non-woven graft materials for use in specialized surgical procedures involving nerve repair and regeneration. Some embodiments describe electrospun fiber products such as conduits, wraps, or grafts comprising a resorbable hybrid-scale matrix to facilitate nerve repair and regeneration.

### CROSS-REFERENCE TO RELATED APPLICATIONS

The present application is a continuation of U.S. patent application Ser. No. 17/903,918, which claims priority to U.S. Provisional Application No. 63/241,366, filed Sep. 7, 2021, which is hereby incorporated by reference in its entirety.

### BACKGROUND

### Field

The present disclosure relates generally to non-woven graft materials, which can be used for example in specialized surgical procedures, such as neurosurgical procedures, wound repair, oral surgery, dermal repair and regeneration, head and neck surgery, endonasal surgery and bone repair, methods for making the non-woven graft materials, and/or methods for repairing tissue such as neurological tissue using the non-woven graft materials.

### SUMMARY

One or more embodiments described herein provide structures having a plurality of aligned (e.g., longitudinally or axially aligned) and non-aligned (e.g. randomly oriented) fibers. When such a structure is used as a biomedical patch, the alignment of fibers as described herein may provide directional cues that influence cell propagation. For example, the structures provided may promote new cell growth along the fibers, such that cell propagation in one or more desired directions may be achieved.

Aspects of the present disclosure relate generally to electrospun resorbable nerve products. In some embodiments, the nerve product may be a conduit, wrap, or graft. In particular, in some embodiments, the present disclosure relates to systems, devices, and methods for the production and application of an electrospun resorbable nerve product.

In some aspects, the present disclosure is directed to a resorbable non-woven graft material comprising: a first non-woven fiber composition, wherein the first fiber composition comprises a polymer selected from the group consisting of polycaprolactone, polydioxanone, poly (glycolic acid), poly(L-lactic acid), poly(lactide-co-glycolide), poly(L-lactide), poly(D,L-lactide), poly(ethylene glycol), montmorillonite, poly(L-lactide-co-ε-caprolactone), poly(ε-caprolactone-co-ethyl ethylene phosphate), poly[bis(p-methylphenoxy) phosphazene], poly(3-hydroxybutyrate-co-3-hydroxyvalerate), poly(ester urethane) urea, poly(p-dioxanone), polyurethane, polyethylene terephthalate, poly(ethylene-co-vinylacetate), poly(ethylene oxide), poly(phosphazene), poly(ethylene-co-vinyl alcohol), polymer nanoclay nanocomposites, poly(ethylenimine), poly(ethyleneoxide), poly vinylpyrrolidone, polystyrene (PS) and combinations thereof; and a second non-woven fiber composition, wherein the second fiber composition comprises a polymer selected from the group consisting of polycaprolactone, polydioxanone, poly (glycolic acid), poly(L-lactic acid), poly(lactide-co-glycolide), poly(L-lactide), poly(D,L-lactide), poly(ethylene glycol), montmorillonite, poly(L-lactide-co-ε-caprolactone), poly(ε-caprolactone-co-ethyl ethylene phosphate), poly[bis(p-methylphenoxy) phosphazene], poly(3-hydroxybutyrate-co-3-hydroxyvalerate), poly(ester urethane) urea, poly(p-dioxanone), polyurethane, polyethylene terephthalate, poly(ethylene-co-vinylacetate), poly(ethylene oxide), poly(phosphazene), poly(ethylene-co-vinyl alcohol), polymer nanoclay nanocomposites, poly(ethylenimine), poly(ethyleneoxide), poly vinylpyrrolidone; polystyrene and combinations thereof; and wherein the first fiber composition and the second fiber composition comprise different polymers.

One or more structures provided may be created using an apparatus that includes one or more first electrodes that define an area and/or partially circumscribe an area. For example, a single first electrode may enclose the area, or a plurality of first electrode(s) may be positioned on at least a portion of the perimeter of the area. A second electrode is positioned within the area. In exemplary embodiments, when the electrodes are electrically charged at a first polarity, and a spinneret dispensing a polymer (e.g., toward the second electrode) is electrically charged at a second polarity opposite the first polarity, the dispensed polymer forms a plurality of fibers extending from the second electrode to the first electrodes. Further, electrodes with rounded (e.g., convex) surfaces may be arranged in an array, and a fibrous structure created using such electrodes may include an array of wells at positions corresponding to the positions of the electrodes

For purposes of this summary, certain aspects, advantages, and novel features of the invention are described herein. It is to be understood that not necessarily all such advantages may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein.

All of these embodiments are intended to be within the scope of the invention herein disclosed. These and other embodiments will become readily apparent to those skilled in the art from the following detailed description having reference to the attached figures, the invention not being limited to any particular disclosed embodiment(s).

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1 depicts embodiments of nerve growth and regeneration patterns based on structured or unstructured underlying material.

FIG. 2 illustrates a set of graphs and cell morphologies for nerve cells.

### DETAILED DESCRIPTION

The detailed description set forth below in connection with the drawings is intended as a description of various configurations and is not intended to represent the only configurations in which the concepts described herein may be practiced. The detailed description include specific details for the purpose of providing a thorough understanding of various concepts. However, it will be apparent to those skilled in the art that these concepts may be practiced without these specific details.

All patents, applications, published applications and other publications referred to herein are incorporated herein by reference to the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the scope of the disclosure as defined by the appended claims.

### Definitions

All technical and scientific terms used herein have the same meaning as commonly understood to one of ordinary skill in the art to which this disclosure belongs unless clearly indicated otherwise.

As used herein, the singular forms “a”, “and”, and “the” include plural referents unless the context clearly dictates otherwise. Thus, for example, reference to “a sequence” may include a plurality of such sequences, and so forth.

The terms comprising, including, containing and various forms of these terms are synonymous with each other and are meant to be equally broad. Moreover, unless explicitly stated to the contrary, examples comprising, including, or having an element or a plurality of elements having a particular property may include additional elements, whether or not the additional elements have that property.

As used herein, the terms “nano-sized fibers” or “nanofibers” refer to very small diameter fibers having an average diameter not greater than 2000 nanometers, and suitably, not greater than 1500 nanometers (nm). Nanofibers are generally understood to have a fiber diameter range of about 10 to about 1500 nm, more specifically from about 10 to about 1000 nm, more specifically still from about 20 to about 500 nm, and most specifically from about 20 to about 400 nm. Other exemplary ranges include from about 50 to about 500 nm, from about 100 to 500 nm, or about 40 to about 200 nm.

As used herein the term “microfibers” refers to small diameter fibers having an average diameter not greater than 75 microns, for example, having an average diameter of from about 0.5 microns to about 50 microns, or more particularly, microfibers having an average diameter of from about 2 microns to about 40 microns. Another frequently used expression of fiber diameter is denier. The diameter of a polypropylene fiber given in microns, for example, may be converted to denier by squaring, and multiplying the result by 0.00629, thus, a 15 micron polypropylene fiber has a denier of about 1.42 (152×0.00629=1.415). As used interchangeably herein, “non-woven graft material” and “non-woven graft fabric” refer to a material having a structure of individual fibers or threads which are interlaid, but not in an identifiable manner as in a knitted fabric. Non-woven graft materials and non-woven graft fabrics can be formed from many processes such as for example, electrospinning processes, meltblowing processes, spunbonding processes, melt-spraying and bonded carded web processes. The basis weight of non-woven graft materials is usually expressed in ounces of material per square yard (osy) or grams per square meter (gsm) and the fiber diameters are usually expressed in nanometers and micrometers (microns). Suitable basis weight of non-woven graft materials of the present disclosure can range from about 50 gsm to about 300 gsm. More suitably, basis weight of non-woven graft materials of the present disclosure can range from about 70 gsm to about 140 gsm. The tensile strength of the non-woven graft material of the present disclosure can range from about 5 Newtons (N) to about 50 Newtons (N), including from about 1 N to about 10 N to about 15 N. The strength of the non-woven graft material of the present disclosure can also be described in terms of suture pull-out strength, which refers to the force at which a suture can be torn from the non-woven graft material. Suitable suture pull-out strength can range from about 1 N to about 5 N.

As used herein, the term “electrospinning” refers to a technology which produces nano-sized fibers referred to as electrospun fibers from a solution using interactions between fluid dynamics and charged surfaces. In general, formation of the electrospun fiber involves providing a solution to an orifice in a body in electric communication with a voltage source, wherein electric forces assist in forming fine fibers that are deposited on a surface that may be grounded or otherwise at a lower voltage than the body. In electrospinning, a polymer solution or melt provided from one or more needles, slots or other orifices is charged to a high voltage relative to a collection grid. Electrical forces overcome surface tension and cause a fine jet of the polymer solution or melt to move towards the grounded or oppositely charged collection grid. The jet can splay into even finer fiber streams before reaching the target and is collected as interconnected small fibers. Specifically, as the solvent is evaporating (in processes using a solvent), this liquid jet is stretched to many times it original length to produce continuous, ultrathin fibers of the polymer. The dried or solidified fibers can have diameters of about 40 nm, or from about 10 to about 100 nm, although 100 to 500 nm fibers are commonly observed. Various forms of electrospun nanofibers include branched nanofibers, tubes, ribbons and split nanofibers, nanofiber yarns, surface-coated nanofibers (e.g., with carbon, metals, etc.), nanofibers produced in a vacuum, and so forth. The production of electrospun fibers is illustrated in many publication and patents, including, for example, P. W. Gibson et al., “Electrospun Fiber Mats: Transport Properties,” AIChE Journal, 45 (1): 190-195 (January 1999), which is hereby incorporated herein by reference.

As used herein, the term “type” such as when referring to “different types of fibers” or “distinct types of fibers” refers to fibers having “a substantially different overall material composition” with measurably different properties, outside of “average diameter” or other “size” differences. That is, two fibers can be of the same “type” as defined herein, yet have different “average diameters” or “average diameter ranges.” Although fibers are of different “types” when they have a substantially different overall material composition, they can still have one or more components in common. For example, electrospun fibers made from a polymer blend with a first polymeric component present at a level of at least 10 wt % would be considered a different fiber type relative to electrospun fibers made from a polymer blend that was substantially free of the first polymeric component. Fibers of different “types” can also have a completely different content, each made of a different polymer for example, or one made from a polymer fiber and the other from a titania fiber, or a ceramic fiber and a titania fiber, and so on.

As used herein the term “polymer” generally includes but is not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configuration of the material. These configurations include, but are not limited to isotactic and atactic symmetries.

The fiber compositions used in the present disclosure can be produced from any resorbable material that meets the criteria of that material as those criteria are described above. The fiber compositions can be formed from resorbable polymers such as (but not limited to) polymers of lactic and glycolic acids, copolymers of lactic and glycolic acids, poly(ether-co-esters), poly(hydroxybutyrate), polycaprolactone, copolymers of lactic acid and ε-aminocapronic acid, lactide polymers, copolymers of poly(hydroxybutyrate) and 3-hydroxyvalerate, polyesters of succinic acid, poly(N-acetyl-D-glucosamine), polydioxanone, cross-linked hyaluronic acid, cross-linked collagen, and the like, and combinations thereof. Suitable synthetic polymers can be, for example, polycaprolactone (poly(¿-caprolactone), PCL), polydioxanone (PDO), poly (glycolic acid) (PGA), poly(L-lactic acid) (PLA), poly(lactide-co-glycolide) (PLGA), poly(L-lactide) (PLLA), poly(D,L-lactide) (P(DLLA)), poly(ethylene glycol) (PEG), montmorillonite (MMT), poly(L-lactide-co-ε-caprolactone) (P(LLA-CL)), poly(ε-caprolactone-co-ethyl ethylene phosphate) (P(CL-EEP)), poly[bis(p-methylphenoxy) phosphazene] (PNmPh), poly(3-hydroxybutyrate-co-3-hydroxyvalerate) (PHBV), poly(ester urethane) urea (PEUU), poly(p-dioxanone) (PPDO), polyurethane (PU), polyethylene terephthalate (PET), poly(ethylene-co-vinylacetate) (PEVA), poly(ethylene oxide) (PEO), poly(phosphazenc), poly(ethylene-co-vinyl alcohol), polymer nanoclay nanocomposites, poly(ethylenimine), poly(ethyleneoxide), poly vinylpyrrolidone; polystyrene (PS) and combinations thereof. Particularly suitable polymers include poly(lactic-co-glycolic acid), polydioxanone, polycaprolactone, and combinations thereof. In some embodiments, the fiber compositions can be formed from one or more populations consisting of PEO and PEG.

The fibers for the fiber compositions may be of a variety of sizes as deemed suitable by one skilled in the art for the end purpose of the non-woven graft material. Typically, the fibers have a mean fiber diameter of less than 5 μm, including less than 2 μm, including less than 1.5 μm, and including less than 1.0 μm. For example, in some embodiments, the fibers can have a mean fiber diameter ranging from about 10 nm to about 5 μm, more specifically from about 10 nm to about 1.0 μm, more specifically still from about 20 nm to about 500 nm, and most specifically from about 20 nm to about 400 nm. Other exemplary ranges include from about 50 nm to about 500 nm, from about 100 nm to about 500 nm, and about 40 nm to about 200 nm.

Suitable ratios of the first fiber composition to the second fiber composition resulting in the non-woven graft material can range from about 10 to 1 to about 1 to 10.

### SUMMARY

The present disclosure relates generally to non-woven graft materials, which can be used for example in specialized surgical procedures, such as neurosurgical procedures, wound repair, oral surgery, dermal repair and regeneration, head and neck surgery, endonasal surgery and bone repair, methods for making the non-woven graft materials, and methods for repairing tissue such as neurological tissue using the non-woven graft materials.

Aspects of the present disclosure relate generally to electrospun resorbable nerve products. In some embodiments, the nerve product may be a conduit, wrap, or graft. In particular, in some embodiments, the present disclosure relates to systems, devices, and methods for the production and application of an electrospun resorbable nerve product.

In some embodiments, the present disclosure may be related to electrospun nerve conduits, wraps, or grafts. Electrospun nerve conduits, wraps, or grafts may be comprised out of a plurality of flexible, hollow resorbable poly(lactide-co-e-caprolactone) tubes designed to provide a protective environment for peripheral nerve regeneration after injury and to create a conduit to guide axonal growth across a nerve gap. In some embodiments, the conduits, wraps, or grafts may incorporate longitudinally or axially aligned fibers on the inner lumen of the conduits, wraps, or grafts and randomly aligned fibers on the outer lumen of the conduits, wraps, or grafts. In some embodiments, conduits, wraps, or grafts may be constructed out of poly(lactide-co-e-caprolactone). In some embodiments, the conduits, wraps, or grafts may possess suitable handling and mechanical properties for surgical use. In some embodiments, the conduits, wraps, or grafts may demonstrate a suitable tensile strength for surgical implantation, as well as suitably high suture retention strength to facilitate sutured coaptation to a transected nerve. In some embodiments, the conduits, wraps, or grafts may also possess sufficient wall strength so as not to collapse under tissue compression following implantation.

### INTRODUCTION

Every year, roughly twenty million Americans suffer from peripheral nerve injury caused by trauma and medical disorders, and these nerve injuries result in approximately $150 billion spent in annual health-care dollars in the United States. The majority of peripheral nerve injuries typically occur in the upper limb and are from traumatic causes. Severe nerve injury often leads to sensory and motor function defects that can result in complete paralysis of the affected limb or development of debilitating neuropathic pain. The primary goal of nerve repair can be to facilitate effective regeneration of the injured nerve and reinnervation of distal motor and sensory end organs needed for recovery of sensorimotor function.

In particular, nerve injury can involve one or more transected nerves. When a nerve is transected, surgical repair can often be required. Generally speaking, the greater the gap length between the ends of the transected nerve, the more challenging the repair. Additionally, larger gap lengths can be associated with reduced sensorimotor outcomes. In fact, gap segment length can be the most important factor in deciding which technique or device to use in a repair. For example, severed nerves can be directly repaired if the nerve ends are close together (<8.9 mm gap) and require no tension on the nerve to reconnect. These can be called “short gap” or “no gap” injuries. In some instances, repairs often include approximating the nerve ending margins with suture. Protective wraps or coaptation aids may be used to strengthen the repair. Given the likely stress on a repaired nerve (from local tissue swelling, vascular supply compromises, flexion/extension of anatomy), the direct repair of damaged nerve ends is typically infrequent and risks poor sensorimotor outcomes. Larger gap nerve injuries can require a “bridging” material to reconnect the transected ends of an injured nerve across the nerve gap. These interventions can be required both in instance of short gap and long gap injuries where the surgeon feels it is too risky to join the nerve endings with suture. Wraps, conduits, and/or grafts, such as some embodiments described herein, may be used in these types of surgeries.

In addition, nerve injury can also involve crushed and/or compressed nerves. In injuries where the nerve is not transected and the nerve ultrastructure is still intact, surgical repair is typically less common. However, in severe cases, surgery may be needed to reduce pressure and inflammation while protecting the nerve. Nerve wraps, such as those described herein, may be utilized to protect the nerve while it heals, providing a barrier from surrounding tissue. Compression injuries, where blood supply and nerve structure in still intact, can have a greater likelihood of complete sensorimotor recovery.

Of the injuries that require surgical nerve reconstruction, approximately 560K can require and/or benefit from the use of nerve grafts, wraps or coverings, such as one or more embodiments described herein. 55% of these (˜307K) can be considered “no gap” procedures, where the size of the gap is less than 8.9 mm; and the remainder can be considered gap procedures, resulting in a gap greater than 9 mm.

Generally speaking, there can be three types of biomaterial-based devices for nerve repair. For example, in some embodiments, conduits are comprised of collagen or synthetic materials and can be used to connect transected nerve ends and guide regenerating nerve fibers from the proximal nerve stump to the distal nerve stump. These devices can be deployed by grossly aligning the fascicles in the proximal and distal nerve stumps and then suturing the nerve stumps in to either end of the conduit, thereby eliminating potential stress on the nerve. In some embodiments, conduits are used for very short gaps (20 mm or less) due to the lack of structural support for regenerating axons and the inability of conduits to successfully bridge larger nerve gaps. In addition, in some embodiments, wraps can be used to protect the nerve while healing, provide a barrier from surrounding tissue, or to strengthen the repair. Moreover, in some embodiments, allografts can be human cadaveric nerve tissue that has been chemically processed to remove all donor cells and then terminally sterilized. The processed acellular nerve allograft can be used to repair and reconnect the damaged nerve by suturing the graft end to end with the transected nerve stumps.

However, peripheral nerve injuries are challenging clinical problems that commonly result in permanent sensorimotor deficits despite the use of the most advanced surgical interventions. In other words, current products and/or clinical approach may not fully address clinical needs, the shortcomings of which some embodiments described herein address.

In particular, microsurgical repair of injured nerves utilizing the patient's own harvested nerve tissue can represent the clinical gold-standard; however, this can be limited by graft availability and permanent nerve damage at the site of graft harvest. Furthermore, autograft repair may only support substantial sensorimotor recovery in roughly 50% of patients, particularly those with small gap rather than large gap injuries. Apart from the patient's own harvested nerve tissue, few clinical options currently exist for repairing damage nerve. Multiple synthetic and xenogenic nerve conduits and wraps have been developed but are only effective in repairing small gap injuries, unlike some embodiments described herein. In particular, existing decellularized nerve allograft products, while showing improvement over existing nerve conduits, demonstrated questionable performance in long gap settings. As a result, new and improved solutions for peripheral nerve repair capable of improving the speed and quality of functional nerve regeneration in both small and large gap injuries are needed. Some embodiments described herein address such technical shortcomings and needs.

Generally speaking, small and large gap injuries are typically most commonly repaired utilizing harvested nerve autografts due to ability to achieve superior clinical outcomes. Autograft repair can involve harvesting a nerve from the patient's own body (often the leg), thereby requiring 2 surgeries/incisions and inducing donor site morbidity at the harvest site. This donor nerve may be utilized directly to repair the nerve defect by suturing the graft end to end with the transected nerve stumps, or in cases involving repair of larger diameter nerves, may be bundled together to create a larger graft for use in repairing the damaged nerve.

Typically, human autografts can be preferred as autografting can be superior to nerve conduits for longer gaps (>3 cm), more proximal injuries, and critical nerves. Autograft repair can represent the current gold-standard treatment for peripheral nerve injuries and offers a 50.5-81.6% recovery rate. However, donor site morbidity at the site of nerve harvest can result in permanent sensory deficits.

In some embodiments, nerve grafts can be single, cable, trunk, interfascicular, or vascularized. In some embodiments, a single graft can join nerve gaps with a segment of a donor nerve of similar diameter. In some embodiments, to span gaps between large diameter nerves, cable grafts can be used, comprising multiple lengths of a smaller diameter donor nerve to approximate the diameter of the injured nerve. In some embodiments, donor nerve grafts are harvested from expendable sensory nerves including the sural and medial antebrachial and are reversed in orientation to maximize the number of axons successfully regenerating through the graft by funneling them distally. In some embodiments, this can prevent loss of regenerating axons downside branches of the donor nerve graft.

In some instances, human cadaveric nerve allografts can be used in a limited number of patients with extensive nerve injuries and inadequate autologous nerve donor tissue. In some embodiments, compared to autografts there are no donor supply limitations or donor site morbidity; however, there can be significant costs and complexity with their use. For example, early human nerve allografts were used without decellularization. In these cases, donor Schwann cells within the nerve allografts may display major histocompatibility complexes and incited a T-cell response. Therefore, recipients may be immunosuppressed for up to two years until the donor nerve graft has been repopulated with host Schwann cells.

In some embodiments, recent advancement in decellularization techniques employing chemical detergents, enzymatic degradation, and irradiation can allow for the production of decellularized nerve allografts with no requirements for immunosuppression. For example, a nerve allograft product can be configured for use in short and long graft repair scenarios. In some embodiments, the processed and sterilized cadaveric nerve allografts preclude the need for autologous nerve harvesting, increasing off-the-shelf availability of nerve graft material and eliminating donor site morbidity. Allograft repair can demonstrate positive clinical outcomes particularly in small gap injuries <30 mm.

However, in some instances, the acellular nerve allograft can demonstrate inconsistent clinical performance when utilized to repair nerve gaps >30 mm. Unlike nerve autografts, the decellularized nerve allograft can lack autologous cells critical to axonal regeneration. Specifically, in some instances, the absence of autologous Schwann cells, extracellular matrix proteins, and growth factors can reduce the ability of processed nerve allografts to support nerve regeneration over long distances. Clinical studies have demonstrated that sensory, mixed, and motor nerves repaired with short acellular nerve allograft recovered at 88.6%, 77%, and 85.7%, respectively. Further, the efficacy of the graft in various nerve gap lengths and showed that short (5 to 14 mm) recovered at 100%, medium (15 to 29 mm) recovered at 76.2%, and long (30 to 50 mm) recovered at 90.9% (mean follow up 265-279 d). Yet, alternate studies suggest that acellular nerve allografts do not perform equivalent to nerve autograft, particularly in long nerve gap injuries. As a result, surgeon confidence with allografts in long gap repairs remains low and new and improved solutions are needed.

Generally speaking, the advantage of acellular nerve allografts over hollow nerve conduits can be that the internal nerve structure including endoneurial tubes, basal lamina, and laminin remain intact, facilitating axonal regeneration. A recent level III study demonstrated functional recovery for injuries with gaps between 5 and 50 mm. However, the majority of their clinical use has been limited to small sensory nerves, for example, digital nerves, for gaps less than 3 cm. Decellularized nerve grafts or nerve conduits may not be considered a replacement for autologous nerve grafting in motor nerves, gaps more than 3 cm, or in proximal nerve injuries. This is because certain allografts are typically prepared with detergents, ChABC enzyme optimized to remove axon-inhibiting proteins and encourage axonal regeneration. Axonal regeneration can only progress/extend a limited distance with the organized and concerted support of active Schwann cells. Certain allograft may not adequately attract/support Schwann cell infiltration, resulting in an “unsupportive dead zone” in the middle of the graft. There remains a significant need for effective alternatives to nerve autograft repair in the setting of long gap injuries, including those optimized for Schwann cell infiltration. Some embodiments described herein address such technical shortcomings.

In addition, in some embodiments, conduits and/or wraps are provided. Existing conduits have failed to demonstrate equivalent or superior outcomes to autografts for gaps greater than 3 cm. Conduits can be categorized as autogenous biological, non-autogenous biological, or nonbiological. In some embodiments, autogenous biological conduits can include hollow vein and arterial conduits and soft tissues, including muscle and tendon grafts. The concern with muscle grafts is that regenerating axons are not contained within the graft and may form neuromas or aberrant regeneration. Vein conduits are the most popular biological conduits and prospective study of twenty-two patients with defects of <3 cm in the hand and forearm, finding that autogenous vein nerve conduits produced results comparable to sural nerve digital grafts. In some embodiments, the use of vein grafts may be reserved for small, less functional nerves with small nerve gaps (e.g., digital sensory nerves with less than a 3 cm gap).

In addition, in some embodiments, nonautogenous biological conduits are provided. In some embodiments, nonautogenous biological conduits have been made from collagens type I, III, or IV and are available in clinical settings. Animal studies with collagen conduits have demonstrated equivalent efficacy when compared with autograft; however, comprehensive clinical studies are lacking. In some embodiments, second generation resorbable nonbiological conduits are made from polyglycolic acid (PGA), polylactic acid (PLA), or poly lactide-co-glycolide acid (PLGA). In some instances, nonresorbable conduits including silicone and Gore-Tex demonstrated unwanted effects including axonal compression during regeneration and fibrous foreign body reaction. PGA nerve conduits have been assessed by a number of clinical studies and demonstrate equivalent results to nerve repairs or autologous grafts for short or moderate digital nerve gaps.

Extensive research continues to focus on adding internal structure, Schwann cells, and growth factors to support axonal regeneration. A prospective trial compared vein grafts to PGA conduits in sensory nerve gaps of 4-25 mm that demonstrated equivalent cost and sensory outcomes at 12 months. In some instances some autologous nerve graft alternatives including decellularized nerve grafts and autogenous and nonautogenous conduits can demonstrate similar efficacy but their use may be limited to sensory nerves with small gaps <3 cm. Primary nerve repair or autogenous nerve grafts can be used for surgical nerve reconstruction for severe nerve injuries.

In some instances, nerve conduits have demonstrated clinical success in short gap injuries. An analysis of median and ulnar nerve repairs demonstrated that 51.6% achieve satisfactory motor recovery (M4-5), with (42.6%), experiencing satisfactory sensory recovery (S3+ to S4). Younger age and more distal injuries may have better outcomes, although many articles report higher rates of “good” motor outcomes using a lower cutoff (M3—movement against gravity only). In some embodiments, use of synthetic nerve conduits have demonstrated an improvement over early technique. Clinical use of ePTFE conduits in 43 patients exhibiting median and ulnar nerve gaps ranging from 1.5 to 6 cm was reported. Patients with smaller gaps (up to 4 cm) had significantly improved outcomes vs larger gaps (78.6% vs 13.3% functional recovery). In some embodiments, xenogenic collagen conduits were utilized in a prospective series of 22 digital nerve repairs and achieved excellent or good sensory outcomes in only 15 of 22 (68%) of subjects. The xenogenic collagen study only focused on small nerve gaps and excluded nerve gaps greater than 20 mm. In a larger study of 126 nerve injuries in 96 patients, nerve injuries were repaired with a xenogenic collagen conduit with an average nerve gap of 12.8 (range 2.5 to 20 mm). In this cohort, nerve function recovery was only observed in 43% of subjects. In total, these results demonstrate both the opportunity for improvement in the speed and quality of functional recovery both in small gap and large gap injuries. Some embodiments described herein address such technical shortcomings.

### Electrospun Hybrid-Scale Fibers

Electrospun hybrid-scale fibers represent a new class of synthetic nanomaterials with the capability to mimic the hierarchical structure of the extracellular matrix (ECM). Most importantly, electrospun nanofibers can be readily collected as uniaxially aligned arrays, providing topographical cues to direct and enhance axonal extension during regeneration. In some embodiments, by controlling size, alignment, and stacking, scaffolds made of electrospun hybrid-scale fibers can be easily adapted for direct implantation. These unique features can make electrospun hybrid-scale fibers an intriguing class of scaffolds with unique applications in neural tissue engineering. Hybrid-scale nanofibers include fibers at various dimensional scales, including microscale fibers, nanoscale fibers, and other fiber populations.

### Nerve Conduit

In some embodiments, a fully-synthetic nanofiber nerve conduit comprised of two layers of electrospun nanofibers rolled into a tube, with the inner and outer layers containing uniaxially aligned and randomly aligned fibers, respectively is provided. In some embodiments, randomly oriented nanofibers provided an isotropic mechanical property for the NGC (nerve graft conduit) and render the conduit suturable and tear-resistant during surgical implantation. In some embodiments, aligned nanofibers served the role of guiding and supporting regenerating axons into the distal nerve stump via topographical cues. Prior studies demonstrated the success of NGCs comprised of aligned nanofibers to enhance axonal regeneration, yet also demonstrated the need to enhance the handling and suturability of the graft through the addition of a reinforcing layer of nanofibers. In some instances, random fibers were added to the outside of the aligned fiber layer to provide additional mechanical support without compromising the guidance cues presented to the axons extending through the inner lumen of the conduit. In some embodiments, the bi-layer electrospun construction, and integration of aligned nanofibers presenting topographical contact cues may enhance overall neuronal repair.

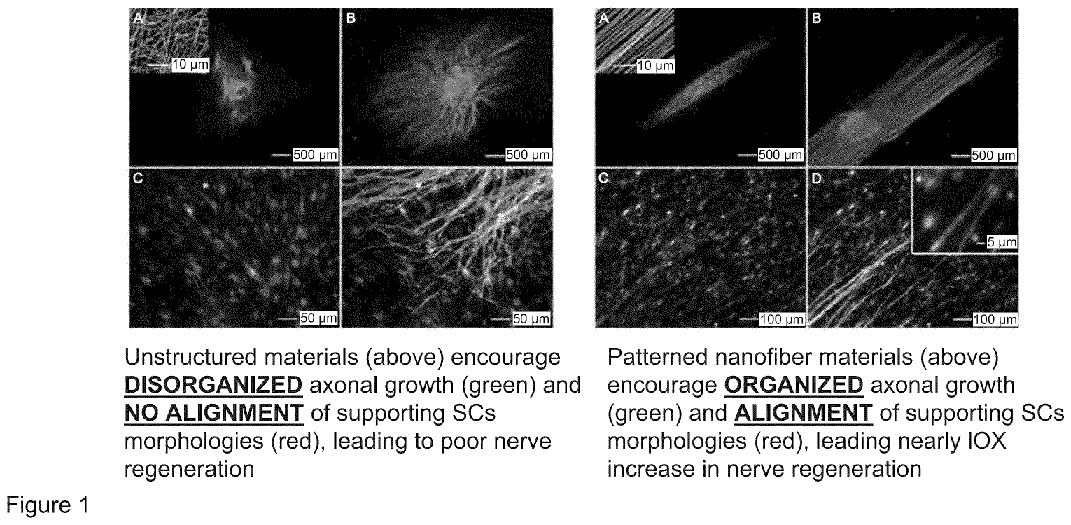

In some embodiments, in order to demonstrate the ability of a bi-layer nanofiber conduit to enhance and direct both axonal regeneration and Schwann cell migration a cell culture model was utilized (FIG. 1). Bundles of neurons, known as dorsal root ganglia (DRG), were utilized as a model system to demonstrate how neurite outgrowth occurs on (i) a pristine double-layered scaffold, with uniaxially aligned and random fibers in the top and bottom layers, respectively, and (ii) a double-layered scaffold pre-seeded with primary Schwann cells, a type of glial cell in the PNS. In some instances, aligned nanofiber matrices supported neurite extension from DRG in both the absence and presence of pre-seeded Schwann cells. In both cases extending neurites were observed to project from the neuronal cell bodies and run parallel to the electrospun fibers on the inner surface of the scaffold. In some instances, this behavior is significantly different than that of cell bodies placed on randomly oriented nanofiber scaffolds, and demonstrates that aligned nanofiber are able to spatially guide extending axons along the length of the conduit toward the distal nerve stump. In some embodiments, the studies also demonstrated that the aligned nanofiber were also able to support and orient Schwann cells, a critical supporting cell type necessary for successful nerve regeneration. FIG. 1 illustrates neuronal growth patterns on a variety of substrates. Unstructured materials encourage disorganized axonal growth with no or little alignment of supporting schwann cells (SCs). In comparison, patterned nanofiber materials encouraged organized axonal growth and alignment of supporting SCs, leading to nearly 10× increase in nerve regeneration. In some embodiments, successful seeding of Schwann cells on to the surface of the nanofiber conduit demonstrate the ability of the conduit to support Schwann cell infiltration and further enhance axonal elongation through the conduit. In some instances, in vitro studies confirmed the ability of the nanofiber conduit to support neurite outgrowth, direct and enhance axonal elongation, and support Schwann cell infiltration capable of further enhancing nerve regeneration.

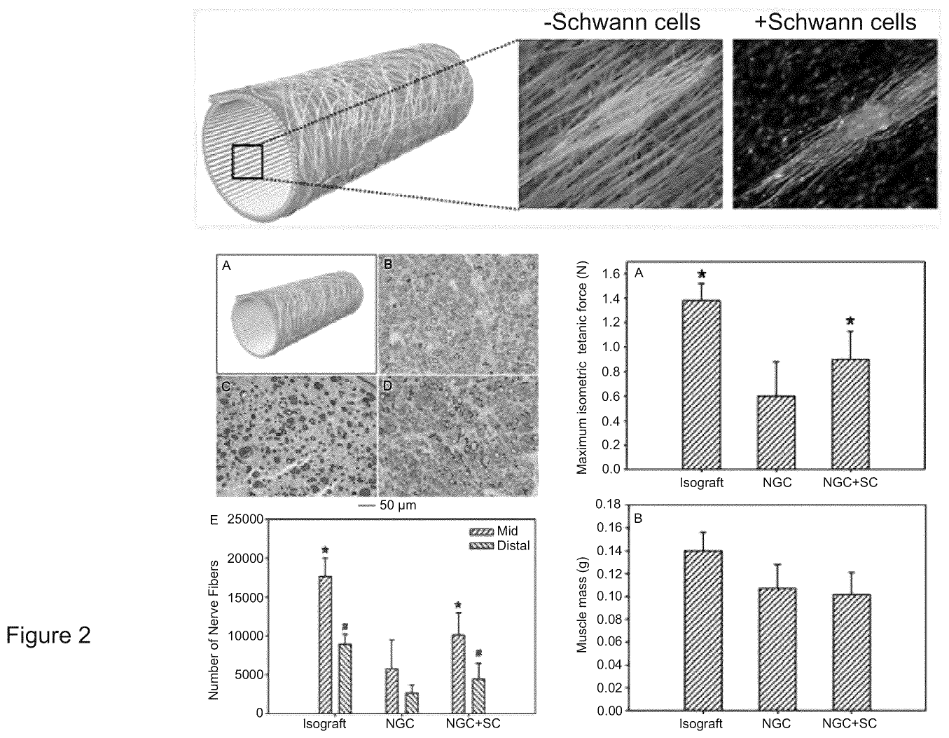

In some instances, an in vivo sciatic nerve transaction/repair model was utilized. Rats were anesthetized prior to surgical exposure of the right sciatic nerve through a dorsolateral gluteal muscle-splitting incision. The sciatic nerve was transected 5 mm proximal to the trifurcation to create a critical 14 mm nerve gap injury. The sciatic nerve was then repaired with either a 14 mm reversed nerve autograft (Isograph), a 16 mm electrospun nanofiber conduit (NGC), or a 16 mm electrospun nanofiber conduit pre-seeded with Schwann cells (NGC+SC) (See FIG. 2). Nerve grafts and conduits were microsurgically sutured to the proximal and distal nerve stumps using one 10-0 nylon suture and secured with fibrin sealant. Recipient nerves in all groups were challenged with a consistent 14 mm nerve gap. Following implantation, muscle fascia and skin were closed in two layers and animals were allowed to recover. Twelve weeks post-operatively, all animals were anesthetized and in situ electrophysiological testing of the regenerated nerve was performed. Additionally, functional recovery was assessed via measurement of evoked muscle force distal to the nerve repair site. Following testing, all animals were euthanized and the entire sciatic nerve was explanted for histomorphometric analysis and assessment of the quality of regenerated nerve tissue. In some embodiments, the in vivo testing demonstrated that the electrospun nanofiber conduit successfully repaired the critical nerve injury in the rodent nerve and facilitated successful reinnervation of distal musculature (FIG. 2). Histomorphological examination of regenerated nerve segments further demonstrated that the electrospun nanofiber conduits successfully supported axonal regeneration across the nerve gap and into the distal nerve stump. In some instances, the electrospun nanofiber conduit pre-seeded with autologous Schwann cells also highlights the future potential of the device to serve as a vehicle for pro-regenerative factors, such as chemotrophic growth factors (GDNF, BDNF, NGF, etc.) or stem cells.

In some embodiments, presented herein is an electrospun nerve conduit device comprised of fully resorbable polymeric fibers. In some embodiments, the electrospun nerve conduit device comprises a hollow tube of various diameters and lengths. In some embodiments, the hollow tube may comprise an inner and outer surface. In some embodiments, the inner surface of the tube may be comprised of longitudinally aligned hybrid-scale fibers structures which may direct and enhance nerve fiber regeneration and elongation. In some embodiments, aligned fiber matrices may improve axonal elongation up to 800% over conventional unorganized matrices. In some embodiments, the outer layer of the conduit may be comprised of randomly organized fibers in order to provide adequate suture strength and handling properties. In some embodiments, the electrospun nerve conduit device may be provided in terminally sterilized in a double peel foil pouches. In some embodiments, the electrospun nerve conduit device comprise a blend of PCL and PGA. In some embodiments, the aligned hybrid-scale fibers structures may increase the rate of infiltration of supporting cell types. In some embodiments, the supporting cell types are Schwann cells. In some embodiments, the electrospun synthetic nerve graft conduit is shelf stable. In some embodiments, no secondary site morbidity exists when the electrospun synthetic nerve conduit device is applied.

In some embodiments, the electrospun nerve conduit device may possess an inner diameter of 1.5 mm, with a length of 24 mm. In some embodiments, the electrospun nerve conduit device may possess an inner Core Length of 20 mm, such that there is a 2 mm Outer Lumen overhang on either side. In some embodiments, the geometry of the electrospun nerve conduit device may be tubular. In some embodiments, the fiber alignment of the electrospun nerve conduit device may comprise longitudinally or axially aligned fibers on the inner lumen of the tube, and randomly aligned fibers on the outer lumen of the tube. In some embodiments, the total wall thickness may range from 325 microns to 550 microns. In some embodiments, the outer lumen is comprised of randomly aligned fibers. In some embodiments, the outer lumen should represent at least 75% of the cross-sectional thickness. In some embodiments, the mean fiber diameter is less than 2 microns. In some embodiments, the mean pore area of the outer lumen is less than 300 microns². In some embodiments of the electrospun nerve conduit device, resorption and mass loss is 50% at 24 weeks. In some embodiments of the electrospun nerve conduit device, resorption and mass loss is 100% at 50 weeks. In some embodiments of the electrospun nerve conduit device, strength loss is 100% at 10 weeks. In some embodiments, the mean tensile strength is greater than 15 newtons. In some embodiments, the minimum tensile strength is greater than 10.5 newtons. In some embodiments, the mean suture retention strength is greater than 3 newtons. In some embodiments, the minimum suture retention strength may be greater than 1 newton. In some embodiments, the electrospun nerve conduit device may withstand compressive forces greater than 0.25 newtons without collapsing.

### Nerve Wrap

In some embodiments, presented herein is an electrospun nerve wrap device comprised of fully resorbable polymeric fibers. In some embodiments, the electrospun nerve wrap device comprises a hollow tube of various diameters and lengths. In some embodiments, the hollow tube may comprise an inner and outer surface. In some embodiments, the inner surface of the tube may be comprised of longitudinally aligned hybrid-scale fibers structures which may direct and enhance nerve fiber regeneration and elongation. In some embodiments, aligned fiber matrices may improve axonal elongation up to 800% over conventional unorganized matrices. In some embodiments, the outer layer of the wrap may be comprised of randomly organized fibers in order to provide adequate suture strength and handling properties. In some embodiments, the electrospun nerve wrap device may be provided in terminally sterilized in a double peel foil pouches. In some embodiments, the electrospun nerve wrap device comprise a blend of PCL and PGA. In some embodiments, the aligned hybrid-scale fibers structures may increase the rate of infiltration of supporting cell types. In some embodiments, the supporting cell types are Schwann cells. In some embodiments, the electrospun nerve wrap device may comprise a slit cut along the outer surface. In some embodiments, the wrap may be utilized to protect and insure coaptation sites between the native nerve or donor nerve or nerve conduits or nerve grafts. In some embodiments, the electrospun nerve wrap device is placed on the nerve coaptation site after suturing or fixation has occurred. In some embodiments, the electrospun nerve wrap device may be wrapped around the nerve to protect the suture/fixation site and guide extending nerve fibers into the distal nerve stump and prevent axons from growing out into surrounding tissue. In some embodiments, the electrospun synthetic nerve wrap device is shelf stable. In some embodiments, no secondary site morbidity exists when the electrospun synthetic nerve wrap device is applied.

In some embodiments, the electrospun nerve wrap device may possess an inner diameter of 1.5 mm, with a length of 24 mm. In some embodiments, the electrospun nerve wrap device may possess an inner Core Length of 20 mm, such that there is a 2 mm Outer Lumen overhang on either side. In some embodiments, the geometry of the electrospun nerve wrap device may be tubular. In some embodiments, the fiber alignment of the electrospun nerve wrap device may comprise longitudinally or axially aligned fibers on the inner lumen of the tube, and randomly aligned fibers on the outer lumen of the tube. In some embodiments, the total wall thickness may range from 325 microns to 550 microns. In some embodiments, the outer lumen is comprised of randomly aligned fibers. In some embodiments, the outer lumen should represent at least 75% of the cross-sectional thickness. In some embodiments, the mean fiber diameter is less than 2 microns. In some embodiments, the mean pore area of the outer lumen is less than 300 microns². In some embodiments of the electrospun nerve wrap device, resorption and mass loss is 50% at 24 weeks. In some embodiments of the electrospun nerve wrap device, resorption and mass loss is 100% at 50 weeks. In some embodiments of the electrospun nerve wrap device, strength loss is 100% at 10 weeks. In some embodiments, the mean tensile strength is greater than 15 newtons. In some embodiments, the minimum tensile strength is greater than 10.5 newtons. In some embodiments, the mean suture retention strength is greater than 3 newtons. In some embodiments, the minimum suture retention strength may be greater than 1 newton. In some embodiments, the electrospun nerve wrap device may withstand compressive forces greater than 0.25 newtons without collapsing.

### Nerve Graft

In some embodiments, presented herein is an electrospun synthetic nerve graft device comprised of a 3D matrix of aligned resorbable polymeric fibers. In some embodiments, the electrospun synthetic nerve graft device comprises a cylinder or cable, available in various diameters and lengths. In some embodiments, the electrospun synthetic nerve graft device is comprised polymeric fibers and mimics the aligned structure and architecture of native nerve tissue. In some embodiments, the outer surface of the electrospun synthetic nerve graft device may be comprised of a layer of randomly organized fibers that will mimic the epineurium and provide adequate suture strength and handling properties. In some embodiments, the cross-sectional area of the electrospun synthetic nerve graft device may be composed of a 3D matrix of highly-porous, longitudinally aligned resorbable hybrid-scale fibers. In some embodiments, the electrospun synthetic nerve graft device may mimic the structure and architecture of endoneurial tubes. In some embodiments, the electrospun synthetic nerve graft device is shelf stable. In some embodiments, no secondary site morbidity exists when the electrospun synthetic nerve graft device is applied.

In some embodiments, presented herein is an electrospun nerve graft device comprised of fully resorbable polymeric fibers. In some embodiments, the electrospun nerve graft device comprises a hollow tube of various diameters and lengths. In some embodiments, the hollow tube may comprise an inner and outer surface. In some embodiments, the inner surface of the tube may be comprised of longitudinally aligned hybrid-scale fibers structures which may direct and enhance nerve fiber regeneration and elongation. In some embodiments, aligned fiber matrices may improve axonal elongation up to 800% over conventional unorganized matrices. In some embodiments, the outer layer of the graft may be comprised of randomly organized fibers in order to provide adequate suture strength and handling properties. In some embodiments, the electrospun nerve graft device may be provided in terminally sterilized in a double peel foil pouches. In some embodiments, the electrospun nerve graft device comprise a blend of PCL and PGA. In some embodiments, the aligned hybrid-scale fibers structures may increase the rate of infiltration of supporting cell types. In some embodiments, the supporting cell types are Schwann cells.

In some embodiments, the electrospun nerve graft device may possess an inner diameter of 1.5 mm, with a length of 24 mm. In some embodiments, the electrospun nerve graft device may possess an inner Core Length of 20 mm, such that there is a 2 mm Outer Lumen overhang on either side. In some embodiments, the geometry of the electrospun nerve graft device may be cylindrical. In some embodiments, the fiber alignment of the electrospun nerve graft device may comprise longitudinally or axially aligned fibers on the inner lumen of the tube, and randomly aligned fibers on the outer lumen of the cylinder. In some embodiments, various densities may be represented throughout the longitudinally or axially aligned fibers throughout the cross section of the cylinder. In some embodiments, the cylinder may comprise three regions of differing densities of fibers. In some embodiments, the total wall thickness may range from 250 microns to 400 microns. In some embodiments, the outer lumen is comprised of randomly aligned fibers. In some embodiments, the outer lumen should represent at least 75% of the cross-sectional thickness. In some embodiments, the mean fiber diameter is less than 2 microns. In some embodiments, the mean pore area of the outer lumen is less than 300 microns². In some embodiments of the electrospun nerve graft device, resorption and mass loss is 50% at 24 weeks. In some embodiments of the electrospun nerve graft device, resorption and mass loss is 100% at 50 weeks. In some embodiments of the electrospun nerve graft device, strength loss is 100% at 10 weeks. In some embodiments, the mean tensile strength is greater than 15 newtons. In some embodiments, the minimum tensile strength is greater than 10.5 newtons. In some embodiments, the mean suture retention strength is greater than 3 newtons. In some embodiments, the minimum suture retention strength may be greater than 1 newton. In some embodiments, the electrospun nerve graft device may withstand compressive forces greater than 0.25 newtons without collapsing.

### Example Embodiments

In some embodiments, any of the following arrangements are contemplated herein:

A resorbable hybrid-scale fiber matrix for use in facilitating nerve repair and regeneration.

In some embodiments, the resorbable hybrid-scale fiber matrix having a shape/geometry selected from a group of consisting of a tube, a conduit, a graft, a wrap, or a spiral wrap.

In some embodiments, the resorbable hybrid-scale fiber matrix being comprised of hybrid-scale fibers composed of a resorbable polymer selected from a group of polymers consisting of polycaprolactone, polylactic acid, polyglycolic acid, polydioxanone, poly(3-hydroxybutyrate-co-3-hydroxyvalerate), polyethylene oxide, polyethylene glycol, and combinations thereof.

In some embodiments, the resorbable hybrid-scale fiber matrix being comprised of hybrid-scale fibers composed of biologically derived and natural materials selected from a group of materials consisting of collagen, elastin, laminin, fibrin, etc.

In some embodiments, the resorbable hybrid-scale fiber matrix wherein the hybrid-scale fibers are formed via method of manufacturing selected from a group of methods consisting of electrospinning, melt electrospinning, force spinning, melt blowing, solvent casting, or combinations thereof.

In some embodiments, the resorbable hybrid-scale fiber matrix wherein a portion of the hybrid-scale fibers are uniaxially or longitudinally aligned, and a portion of the hybrid-scale fibers are randomly aligned.

In some embodiments, the resorbable hybrid-scale fiber matrix wherein the matrix is comprised of 2 or more layers of hybrid-scale fibers.

In some embodiments, the resorbable hybrid-scale fiber matrix wherein the hybrid-scale fibers possess a range of fiber diameters between 10 nm-10,000 nm.

In some embodiments, the resorbable hybrid-scale fiber matrix wherein the diameters of the hybrid-scale fibers are distributed in a bimodal distributions with at least 25% of fibers possessing a diameter <1000 nm and at least 25% of fibers possessing a diameter >1000 nm.

In some embodiments, the resorbable hybrid-scale fiber matrix wherein the diameters of the hybrid-scale fibers are distributed in a bimodal distributions with at least 10% of fibers possessing a diameter <1000 nm and at least 10% of fibers possessing a diameter >1000 nm.

In some embodiments, the resorbable hybrid-scale fiber matrix wherein the mean pore size is less than 300 μm².

In some embodiments, the resorbable hybrid-scale fiber matrix wherein the porosity of the hybrid-scale fibers . . . and the mean pore size is less than 500 μm².

In some embodiments, the resorbable hybrid-scale fiber matrix wherein the hybrid scale fibers are designed to progressively resorb over time in parallel with tissue regeneration and formation between 4-54 weeks.

In some embodiments, the resorbable hybrid-scale fiber matrix wherein the hybrid scale fibers are designed to progressively resorb over time in parallel with tissue regeneration and formation between 4-100 weeks.

In some embodiments, the resorbable hybrid-scale fiber matrix wherein the wall thickness lies between 250 microns and 550 microns.

In some embodiments, the resorbable hybrid-scale fiber matrix wherein the wall thickness lies between 100 microns and 600 microns.

In some embodiments, the resorbable hybrid-scale fiber matrix wherein the thickness of aligned hybrid-scale fibers lies between 0 to 25% of the total wall thickness.

In some embodiments, the resorbable hybrid-scale fiber matrix wherein the thickness of aligned hybrid-scale fibers lies between 0 to 50% of the total wall thickness.

In some embodiments, the resorbable hybrid-scale fiber matrix wherein the tensile strength should be minimum 10.5 N with a mean greater than 15 N, suture pullout strength should be minimum IN with a mean greater than 3 N, and compressive strength should be greater than 0.25N without collapsing.

In some embodiments, the resorbable hybrid-scale fiber matrix wherein the tensile strength should be minimum 5 N with a mean greater than 10 N, suture pullout strength should be minimum IN with a mean greater than 2 N, and compressive strength should be greater than 0.2 N without collapsing.

In some embodiments, the resorbable hybrid-scale fiber matrix wherein the uniaxially aligned hybrid are positioned circumferentially or throughout the around the cross-sectional area of the matrix.

In some embodiments, the resorbable hybrid-scale fiber matrix wherein inner diameter is less than 2 microns, and the outer diameter is between 1.5 mm-10 mm

In some embodiments, the resorbable hybrid-scale fiber matrix wherein the length of the matrix is 8 mm to 30 mm.

In some embodiments, the resorbable hybrid-scale fiber matrix wherein the length of the matrix is 5 mm to greater than 30 mm.

In some embodiments, the resorbable hybrid-scale fiber matrix wherein the volumetric density of uniaxially aligned fibers is between 0-25%.

In some embodiments, the resorbable hybrid-scale fiber matrix wherein the volumetric density of uniaxially aligned fibers is between 0-50%.

In some embodiments, the resorbable hybrid-scale fiber matrix wherein the volumetric density of uniaxially aligned fibers is low

In some embodiments, the resorbable hybrid-scale fiber matrix wherein the volumetric density of uniaxially aligned fibers is medium

In some embodiments, the resorbable hybrid-scale fiber matrix wherein the volumetric density of uniaxially aligned fibers is high

In some embodiments, the resorbable hybrid-scale fiber matrix wherein the overlap of the outer lumen is between 1-10 mm.

In some embodiments, the resorbable hybrid-scale fiber matrix wherein the matrix is designed to release one or more therapeutically active molecules/biological therapeutics via surface functionalization, bulk loading, physical entrapment, progressive degradation over a defined period of time.

A method for repairing injured nervous tissue by applying the resorbable hybrid-scale fiber matrix to the site of nerve injury.

The method for repairing injured nervous tissue, wherein the injured nerve is identified and surgically exposed or isolated.

The method for repairing injured nervous tissue, wherein the matrix is secured to an injured nerve using one or more techniques including suturing, tissue adhesive, or gravity.

The method for repairing injured nervous tissue, wherein the matrix is applied around the injured nerve tissue.

The method for repairing injured nervous tissue, wherein the matrix is secured end-to-end or in-line with injured nerve tissue.

The method for repairing injured nervous tissue, wherein the matrix supports cellular infiltration, axonal regeneration, elongation, reformation of nervous tissue, and reinnervation of distal nerve segment and end organs.

The method for repairing injured nervous tissue, wherein the matrix completely resorbs via hydrolysis or enzymatic degradation following successful nerve regeneration and functional recovery.

The method for repairing injured nervous tissue, wherein the injured nerve consists of peripheral nerve.

A method for forming a resorbable hybrid-scale fiber matrix for use in facilitating nerve repair and regeneration

The method for forming a resorbable hybrid, wherein the method utilizes a solvent consisting of HFIP, DMF, DCM, Acetone, Chloroform, THF, Acetic Acid, Formic Acid, Trifluoroethanol, or Ethyl acetate.

The method for forming a resorbable hybrid, wherein the method involves forming a first tubular structure from at least two or more layers of hybrid-scale fibers.

The method for forming a resorbable hybrid, wherein the method involves cutting the tubular structure to for a wrap or spiral wrap.

The method for forming a resorbable hybrid, wherein the method involves inserting hybrid-scale fibers into the inner lumen/cross sectional area of a first tubular structure.

The method for forming a resorbable hybrid, wherein the method involves rolling a sheet of hybrid-scale fibers into a tubular or spiral structure.

### Additional Embodiments

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some examples, the actual steps taken in the processes illustrated and/or disclosed may differ from those shown in the figures. Depending on the example, certain of the steps described above may be removed or others may be added. Furthermore, the features and attributes of the specific examples disclosed above may be combined in different ways to form additional examples, all of which fall within the scope of the present disclosure.

For purposes of this disclosure, certain aspects, advantages, and novel features are described herein. Not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without user input or prompting, whether these features, elements, and/or steps are included or are to be performed in any particular example.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

The scope of the present disclosure is not intended to be limited by the specific disclosures of preferred examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

Although the foregoing invention has been described in terms of certain preferred embodiments, other embodiments will be apparent to those of ordinary skill in the art. Additionally, other combinations, omissions, substitutions and modification will be apparent to the skilled artisan, in view of the disclosure herein. Accordingly, the present invention is not intended to be limited by the recitation of the preferred embodiments, but is instead to be defined by reference to the appended claims. All references cited herein are incorporated by reference in their entirety.

The terminology used in the description presented herein is not intended to be interpreted in any limited or restrictive manner and unless otherwise indicated refers to the ordinary meaning as would be understood by one of ordinary skill in the art in view of the specification. Furthermore, embodiments may comprise, consist of, consist essentially of, several novel features, no single one of which is solely responsible for its desirable attributes or is believed to be essential to practicing the embodiments herein described. As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

Although this disclosure is in the context of certain embodiments and examples, those of ordinary skill in the art will understand that the present disclosure extends beyond the specifically disclosed embodiments to other alternative embodiments and/or uses of the embodiments and obvious modifications and equivalents thereof. In addition, while several variations of the embodiments have been shown and described in detail, other modifications, which are within the scope of this disclosure, will be readily apparent to those of ordinary skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes or embodiments of the disclosure. Thus, it is intended that the scope of the present disclosure herein disclosed should not be limited by the particular disclosed embodiments described above.

1. (canceled)

2. A method for facilitating nerve repair and regeneration, the method comprising:
identifying injured nerve tissue in a patient;
surgically isolating the injured nerve tissue;
inserting a hybrid-scale fiber matrix to a target site; and
securing the hybrid-scale fiber matrix to the injured nerve tissue.

3. The method of claim 2, further comprising releasing one or more therapeutically active molecules or biological therapeutics via one or more of surface functionalization, bulk loading, physical entrapment, or progressive degradation over a defined period of time.

4. The method of claim 2, further comprises applying a solvent comprising at least one of HFIP, DMF, DCM, Acetone, Chloroform, THF, Acetic Acid, Formic Acid, Trifluoroethanol, or Ethyl acetate.

5. The method of claim 2, wherein the hybrid-scale fiber matrix comprises an inner fiber layer comprising uniaxially-aligned or longitudinally-aligned fibers and an outer fiber layer comprises randomly-aligned fibers, wherein the hybrid-scale fiber matrix comprises hybrid-scale fibers comprising fibers having a range of fiber diameters between 10 nm and 10,000 nm.

6. The method of claim 2, wherein the hybrid-scale fiber matrix is secured to the injured nerve tissue using one or more techniques including suturing, tissue adhesive, or gravity.

7. The method of claim 2, wherein the hybrid-scale fiber matrix for repairing injured nervous tissue, wherein the hybrid-scale fiber matrix is secured end-to-end or in-line with injured nerve tissue.

8. The method of claim 2, wherein the injured nerve tissue comprises a peripheral nerve.

9. The method of claim 2, wherein the method is used for at least one of wound repair, oral surgery, dermal repair and regeneration, head and neck surgery, endonasal surgery, and bone repair.

10. The method of claim 2, wherein the hybrid-scale fiber matrix comprises a shape geometry selected from a group consisting of a tube, a conduit, a graft, a wrap, or a spiral wrap.

11. The method of claim 2, wherein the hybrid-scale fiber matrix comprise hybrid-scale fibers comprising a resorbable polymer selected from a group of polymers comprising a combination of polycaprolactone, polylactic acid, polyglycolic acid, polydioxanone, PEO, PEG, and poly(3-hydroxybutyrate-co-3-hydroxyvalerate).

12. The method of claim 2, wherein the hybrid-scale fiber matrix comprise hybrid-scale fibers that comprise biologically derived and natural materials selected from a group of materials comprising at least one of collagen, elastin, laminin, or fibrin.

13. The method of claim 2, wherein the hybrid-scale fiber matrix comprise hybrid-scale fibers that are distributed in a bimodal distribution, wherein at least 25% of the hybrid-scale fibers possessing a diameter less than 1000 nm and at least 25% of fibers possessing a diameter greater than 1000 nm.

14. The method of claim 2, wherein the hybrid-scale fiber matrix comprises a mean pore size less than 300 μm².

15. The method of claim 2, wherein the hybrid-scale fiber matrix comprise hybrid-scale fibers that are configured to progressively resorb over time in parallel with tissue regeneration and formation in a time period between 4 to 100 weeks.

16. The method of claim 5, wherein a total thickness of the inner fiber layer and the outer fiber layer comprises at least one of a range between 250 microns and 550 microns or a range between 100 microns and 600 microns.

17. The method of claim 5, wherein a thickness of the inner fiber layer ranges between 0 to 50% of a total thickness of both the inner fiber layer and the outer fiber layer.

18. The method of claim 2, wherein a tensile strength of the hybrid-scale fiber matrix is at least 10.5 N, a suture pullout strength of the hybrid-scale fiber matrix is at least 1 N, and a compressive strength of the hybrid-scale fiber matrix is greater than 0.25 N.

19. The method of claim 5, wherein the hybrid-scale fiber matrix comprises at least one of:
an inner diameter of the inner fiber layer is less than 2 microns, and an outer diameter of the outer fiber layer is between 1.5 mm and 10 mm,
a length of the hybrid-scale fiber matrix that is between 8 mm to 30 mm,
a length of the hybrid-scale fiber matrix is at least 5 mm,

20. The method of claim 2, wherein the hybrid-scale fiber matrix comprises a volumetric density of uniaxially aligned or longitudinally-aligned fibers that is at least one of: between 0-25%, wherein a mean pore size of the hybrid-scale fiber matrix is less than 300 μm², or between 0-50%.

21. The method of claim 5, wherein the outer fiber layer comprises an outer lumen contacting an outer surface of the inner fiber layer, wherein an overhang defined by a difference in an end of the inner fiber layer and an end of the outer fiber layer is between 1-10 mm.
